(19) United States
(12) Patent Application Publication
(10) Pub. No.: US 2024/0216561 A1
(43) Pub. Date: Jul. 4, 2024

(54) A CONTROL DEVICE FOR CONTROLLING AN ULTRAVIOLET LIGHT-BASED DISINFECTION DEVICE

(71) Applicant: SIGNIFY HOLDING B.V., EINDHOVEN (NL)

(72) Inventors: KARS-MICHIEL HUBERT LENSSEN, VELDHOVEN (NL); ERIK PETRUS JOHANNES MALLENS, 'S-HERTOGENBOSCH (NL); TIES VAN BOMMEL, HORST (NL); BARTEL MARINUS VAN DE SLUIS, EINDHOVEN (NL); DZMITRY VIKTOROVICH ALIAKSEYEU, EINDHOVEN (NL); TOBIAS BORRA, RIJSWIJK (NL)

(21) Appl. No.: 18/563,435

(22) PCT Filed: May 19, 2022
(86) PCT No.: PCT/EP2022/063517

§ 371 Date: Nov. 22, 2023

(30) Foreign Application Priority Data
May 27, 2021 (EP) 21176312.3

(51) Int. Cl. A61L 2/24; A61L 2/10; A61L 9/20; H05B 47/105; H05B 47/175; H05B 47/19

(52) U.S. Cl. CPC A61L 2/24; A61L 2/10; A61L 9/20; H05B 47/105; H05B 47/19; H05B 47/196; A61L 2202/11; A61L 2202/14; A61L 2202/16; A61L 2209/111; A61L 2209/12

(57) ABSTRACT

The invention provides a control device for controlling an ultraviolet light-based disinfection device arranged at a disinfection location, wherein the control device comprises: a user interface comprising a user interface element, wherein the user interface element is configured to control an operational parameter of the ultraviolet light-based disinfection device by providing a degree of control in controlling said operational parameter; a controller configured to: (i) determine a spatial relation of the control device relative to the ultraviolet light-based disinfection device and/or the disinfection location, and to (ii) change the degree of control of the user interface element in controlling said operational parameter based on said spatial relation.

### FIELD OF THE INVENTION

The invention relates to a control device for controlling an ultraviolet light-based disinfection device. The invention further relates a control system. The invention further relates to a corresponding method of controlling an ultraviolet light-based disinfection device with such a control device. The invention further relates to a corresponding computer program product. The invention further relates to a system comprising said control device and the ultraviolet light-based disinfection device.

### BACKGROUND OF THE INVENTION

Societal health is periodically contested by virus outbreaks, such as seasonal symptomatic influenza A/B outbreak, SARS, MERS, COVID-19. Future outbreaks, mutations, epidemics, and pandemics are not excluded. At least partly due to an increasing population, urbanization and people movement, the topic of people health & wellbeing is becoming more and more relevant.

Such developments have clearly risen the demand for disinfection devices. For example, a clear demand exists for ultraviolet (UV) light-based disinfection, amongst others ultraviolet-C(UV-C) based disinfection devices. It is namely known that e.g. UV-C radiation has a strong germicidal effect. This demand is not only observed in the professional domain, but also in the consumer market.

The introduction of UV light-based disinfection devices in the consumer market may have already caused an increased risk to eye injuries at home, because at certain doses and exposure times, the UV light of the disinfection device may become harmful. People should not be present at the disinfection location when the UV light-based disinfection device is active. Such UV light-based disinfection devices are therefore equipped with stringent safety measures.

For example, it is known to switch off an UV light-based disinfection device upon detecting proximity of a person with a proximity sensor. However, it may be a clear disadvantage that such proximity sensors do not always function properly, and may cause false negatives. Further, it may not be desired to leave the control of a UV light-based disinfection device to a sensor alone.

As another example of a safety measure, it is seen that UV light-based disinfection devices may comprise a timer button, which may cause, upon user activation of the timer button, the UV light-based disinfection device to switch on after a certain time delay. The time delay allows an initial user of said UV light-based disinfection device to leave the room, in which said UV light-based disinfection device will become active. However, such time delays may still be prone to undesired situations of the initial user, or other people getting back into proximity of the UV light-based disinfection device. The initial user controlling said UV light-based disinfection device may for example not even be aware of people in said room, hence the resulting dangerous situation, if said initial user is e.g. too far away from the UV light-based disinfection device and/or said room.

All in all, a clear need exists to improve the safety of UV light-based disinfection devices, in particular for domestic (non-professional) users. It would also be more convenient to control the UV light-based disinfection device remotely, with improved safety.

### SUMMARY OF THE INVENTION

It is an object of the invention to provide an improved control device for controlling an UV light-based disinfection device, which at least alleviates the problems and disadvantages mentioned above. Thereto, the invention provides a control device for controlling an ultraviolet light-based disinfection device arranged at a disinfection location, wherein the control device is a portable device, wherein the control device comprises: a user interface comprising a user interface element, wherein the user interface element is configured to control an operational parameter of the ultraviolet light-based disinfection device by providing a degree of control in controlling said operational parameter; a controller configured to: (i) determine a spatial relation of the control device relative to the ultraviolet light-based disinfection device and/or the disinfection location, and to (ii) change the degree of control of the user interface element in controlling said operational parameter based on said spatial relation; wherein the spatial relation is a distance of the control device relative to the disinfection location.

The control device according to the invention is configured to control the ultraviolet (UV) light-based disinfection device that is arranged at the disinfection location. Hence, the control device comprises a user interface. This user interface comprises, or is configured to provide, a user interface element. The user interface element is thereby configured to control an operational parameter of the UV light-based disinfection device by providing a degree of control in controlling said operational parameter. Said degree of control may define an extent, e.g. a range of values or options, to which the user interface element is able to control said operational parameter. Said degree of control may be phrase differently as an initial degree of control.

In examples, the user interface element may be at least one user interface element, wherein each user interface element of the at least one user interface element may control a respective operational parameter of the UV light-based disinfection device. As mentioned, the control device is a portable device.

According to the present invention, the control device also comprises a controller configured to determine a spatial relation of the control device relative to the ultraviolet light-based disinfection device and/or the disinfection location. Said spatial relation may e.g. be the distance of the control device relative to the disinfection location. The controller is furthermore configured to change said degree of control of the user interface element in controlling said operational parameter based on said spatial relation.

On the one hand, it may be a safety risk to allow a user to control the control device when the control device is too close to the ultraviolet light-based disinfection device and/or disinfection location. On the other hand, for the sake of safety, it may be undesired to allow a user to control the control device when the control device is too far away from ultraviolet light-based disinfection device and/or disinfection location, because such a user may control a dangerous device without being near said disinfection device and without being able to assess or monitor the disinfection location.

Therefore, since the degree of control of the user interface element in controlling said operational parameter is made dependent on said spatial relation, the control device according to the present invention may dynamically change the degree of control of the ultraviolet light-based disinfection device, when the control device is too close by or too far away from the UV-disinfection device and/or disinfection location. This renders an improved safety measure, or advantageously introduces a safety constraint to the control device.

Said disinfection location may e.g. be defined as a disinfection space, a room, or a disinfection region, or an enclosure (which, in operation, is illuminated by the UV light-based disinfection device). In aspects, said ultraviolet light-based disinfection device may be arranged at a fixed location. However, in alternative aspects, said ultraviolet light-based disinfection device may be mobile. Hence, the ultraviolet light-based disinfection device may move from one disinfection location to another disinfection location.

Throughout the application, in alternative phrasing, said controlling an operational parameter of the ultraviolet light-based disinfection device may be setting the value of an operational parameter of the ultraviolet light-based disinfection device, and said providing a degree of control in controlling said operational parameter may be changing a degree of control in setting the value of said operational parameter.

As mentioned, said controller may be configured to determine the spatial relation of the control device relative to the ultraviolet light-based disinfection device and/or the disinfection location. Said determining may alternatively be phrased throughout the application as obtaining. Hence, the controller may be configured to obtain said spatial relation. For example, the controller may be configured to receive or to retrieve said spatial relation from a further device, which further device may be remote or external to the control device. The further device may e.g. be a positioning system, a user interaction device, a server, a building management system, a sensor, etc.

Throughout the application, according to the invention, said control device may be a control system, wherein the controller and said user interface may be separate from each other. Thereby, advantages and/or embodiments applying to the control device according to the invention may mutatis mutandis apply to said control system according to the invention.

Hence, the invention may provide, a control system for controlling an ultraviolet light-based disinfection device arranged at a disinfection location, wherein the control system comprises: a user interface comprising a user interface element, wherein the user interface element is configured to control an operational parameter of the ultraviolet light-based disinfection device by providing a degree of control in controlling said operational parameter; a controller configured to: (i) determine a spatial relation of the control device relative to the ultraviolet light-based disinfection device and/or the disinfection location, and to (ii) change the degree of control of the user interface element in controlling said operational parameter based on said spatial relation. The controller and user interface element may thereby be physically separated from each other. The user interface element may e.g. be embodied in a user device, whilst the controller may e.g. be in another device, for example a master controller, a server, a lighting device, or even the ultraviolet light-based disinfection device itself.

As partly mentioned, the spatial relation may be a distance of the control device relative to the disinfection location and/or ultraviolet light-based disinfection device. Said distance may be defined as the shortest distance between the control device and the disinfection location. The disinfection location may thereby for example be at an origin, and the distance (e.g. shortest distance) of the control device relative to the origin may be said distance of the control device relative to the disinfection location.

In a related embodiment, the controller may be configured to reduce the degree of control of the user interface element in controlling said operational parameter when the distance of the control device relative to the disinfection location increases. Phrased differently, in an embodiment, the controller may be configured to gradually reduce the degree of control of the user interface element in controlling said operational parameter when the distance of the control device relative to the disinfection location increases.

Such an embodiment may be advantageous, because the degree of control that the user interface element provides in controlling said operational parameter may be changed when the control device is moving further away from the disinfection location, hence further away from the UV light-based disinfection device arranged at said disinfection location. This reduces the ability of a user associated with the control device to continue controlling the UV light-based disinfection device to an initial degree of control, when said user may be too far away from the UV light-based disinfection device. For safety reasons, it may namely be beneficial, from certain distances relative to the disinfection location onwards, not to allow a user to control all, or reduce control of, the user interface element (e.g. a button) controlling an operational parameter of the UV light-based disinfection device.

In aspects, said gradual reducing may alternatively be phrased as instantaneous reducing throughout the application. Hence, in aspects, the controller may be configured to instantaneously reduce the degree of control of the user interface element in controlling said operational parameter when the distance of the control device relative to the disinfection location increases.

In a related embodiment, the controller may be configured to change the degree of control of the user interface element in controlling said operational parameter to a reduced degree of control when the distance of the control device relative to the disinfection location is above a first threshold distance. Similar advantages are attributed to such an embodiment. As more control of the UV-disinfection device is allowed when the control device, and associated user thereof, are closer to the disinfection location, and less when further away from the disinfection location, it may be prevented that a remote (too far away) user may cause a risk of accidents or dangerous situations at the disinfection location.

The first threshold distance may for example be one of: five meter, ten meter, fifty meter, a hundred meter, one kilometer.

In an opposite embodiment, the controller may be configured to reduce the degree of control of the user interface element in controlling said operational parameter when the distance of the control device relative to the disinfection location decreases. Phrased differently, the controller may be configured to gradually reduce the degree of control of the user interface element in controlling said operational parameter when the distance of the control device relative to the disinfection location decreases.

For safety reasons, it may namely be beneficial not to allow a user to control an operational parameter of the UV light-based disinfection device, which may be considered dangerous for the user associated with the control device, when the control device may be too close to the disinfection location. Such an embodiment is therefore advantageous as well, because the degree of control that the user interface element provides in controlling said operational parameter may be changed when the control device is moving closer towards the disinfection location, hence closer to the UV light-based disinfection device arranged at said disinfection location. The degree of control may e.g. limit the user interface element of the control device to control an operational parameter in a dangerous value range for a user, but only allow to control said operational parameter in a safe value range for a user.

In a related embodiment, the controller may be configured to change the degree of control of the user interface element in controlling said operational parameter to a reduced degree of control when the distance of the control device relative to the disinfection location is below a second threshold distance. Similar advantages are attributed to such an embodiment. As less control of the UV-disinfection device is allowed when the control device, and associated user thereof, is closer to the disinfection location, and more when further away from the disinfection location, it may be prevented that a too close-by user may cause a risk of accidents or dangerous situations at the disinfection location and hence to the user itself. The second threshold distance may for example be one of: one meter, five meter, ten meter.

The invention may also provide a combination of the above. Hence, in an embodiment, the controller may be configured to change the degree of control of the user interface element in controlling said operational parameter to a reduced degree of control when the distance of the control device relative to the disinfection location is above a first threshold distance and/or below a second threshold distance. Thereby, in an embodiment, the second threshold distance is smaller than the first threshold distance. Phrased differently, the second threshold distance may be closer to the disinfection location than the first threshold distance.

The first threshold distance and/or the second threshold distance may in some examples be predefined. Hence, the first threshold distance and/or the second threshold distance may be stored in the controller or a memory associated with the controller, such that the controller may determine the first threshold distance and/or the second threshold distance without needing to receive or retrieve the first threshold distance and/or the second threshold distance from another source.

Alternatively, the controller may obtain, receive, or retrieve said first threshold distance and/or said second threshold distance from another source, such as a further device, or a Building Information Model, which may e.g. store commissioning information where the UV light-based disinfection device is installed, and which safety conditions apply (i.e. the first threshold distance and/or the second threshold distance).

Said reduced degree of control may reduce the extent to which the user interface element is able to control said operational parameter. For example, in an embodiment, the reduced degree of control may comprise limiting the user interface element to a limited set of values or options for controlling said operational parameter.

For example, the degree of control, or phrased differently the initial degree of control, may comprise an initial set of values. This initial set of values may cover e.g. a full range. The reduced degree of control may then be a limited set of values of the initial set of values, such as a subrange of the full range. The values may e.g. be wavelength ranges of light, or intensity values of light.

Similarly, the degree of control may comprise an initial set of options for controlling said operational parameter. For example, a number of options for controlling said operational parameter. The reduced degree of control may then comprise a lesser number of options for controlling said operational parameter. The user interface element may e.g. be a menu providing said number of options, but in the reduced degree of control lesser number of options.

Furthermore, in an embodiment, the reduced degree of control may comprise fully disabling the user interface element in controlling said operational parameter. Such an embodiment may be advantageous, as it provides improved safety by (fully) disabling the user interface element in controlling said operational parameter.

In aspects, said disabling may comprise not processing a user input for controlling said operational parameter, such as a touch input or a voice input. Hence, in an aspect, the reduced degree of control may comprise adapting a processing of a user input resulting from an operation of the user interface element. In aspects, said adapting may be stop processing said user input.

Furthermore, in an embodiment, the reduced degree of control may comprise adapting a graphical representation of the user interface element in controlling said operational parameter. Such an embodiment may be advantageous, as the degree of control of the operational parameter of the UV light-based disinfection device is reduced by adapting the graphical representation of the user interface element in controlling said operational parameter, which adaptation in graphical representation may be a direct representation of how the degree of control is reduced.

For example, a graphical representation of the user interface element may be made transparent, or greyed-out, such that the user interface element becomes less visible and noticeable for interfacing with it. For example, a graphical representation of the user interface element may comprise a strikethrough, indicative that said user interface element is not to be used. Similarly, for example, a graphical representation of the user interface element may comprise an alerting feature, indicative that said user interface element is not to be used.

Furthermore, in an embodiment, the reduced degree of control may comprise adapting a size of the user interface element. For example, the size of the user interface element may be made smaller when the degree of control is reduced.

In an embodiment, the controller may comprise a receiver for receiving a wireless signal; wherein the wireless signal comprises an indicator indicative of the distance of the control device relative to the disinfection location; wherein the controller may be configured to determine a distance of the control device relative to the disinfection location based on said indicator. Said distance may alternatively be relative to the disinfection device.

In a related embodiment, the indicator may comprise at least one of: a signal strength, distance data, location information. Said signal strength may e.g. be RSSI. Said distance data and/or said location information may e.g. be received from a further device, said further device being one of: a remote server, a localization device, a positioning system.

For example, a positioning system may provide the indicator comprising location information, such that the controller of the control device may determine the distance of the control device relative to the disinfection location based on said location information. For example, a localization device, such as a GPS antenna, may provide the controller of the control device with location information or distance data, such that the controller of the control device may determine the distance of the control device relative to the disinfection location based on said location information and/or distance data. The control device may, in aspects, also comprise such a localization device, such as a GPS antenna. Other examples may similarly be envisioned.

In a related embodiment, the receiver may be a radiofrequency receiver, a Li-Fi receiver, a VLC receiver, an IR receiver, a camera, or a microphone. Said radiofrequency receiver may for example communicate over Bluetooth, Wi-Fi, RF, Zig-Bee, Lo-Ra, 4G, 5G and/or UWB.

In an embodiment, the controller may comprise a receiver for receiving a wireless signal; wherein the wireless signal comprises a respective entry indicative of the first threshold distance and/or the second threshold distance, wherein the controller is configured to determine the first threshold distance and/or the second threshold distance based on said respective entry. The wireless signal may thereby be received from the UV light-based disinfection device, but also from e.g. a further device such as a backend server (e.g. of the manufacturer of the UV light-based disinfection device).

Furthermore, the present invention may define said spatial relation differently. Namely, in an embodiment, the spatial relationship may be a line-of-sight between the control device and the disinfection device. Such a line-of-sight means that the control device and the disinfection may be visible to each other in an uninterrupted way. Such a line-of-sight may for example be established by an IR transmitter at the ultraviolet light-based disinfection device and a corresponding IR receiver at the control device, or vice versa in that the IR transmitter is at the control device and IR receiver is at the ultraviolet light-based disinfection device.

Hence, in an embodiment, the spatial relationship may be a line-of-sight between the control device and the disinfection device; wherein the controller may be configured to determine a line-of-sight condition between the control device and the disinfection device, and to change the degree of control of the user interface element in controlling said operational parameter to a reduced degree of control when the line-of-sight condition comprises that no line-of sight is present between the control device and the disinfection device. The embodiments and/or examples provided above wherein the spatial relationship is distance of the control device relative to the disinfection location may mutatis mutandis apply to the spatial relationship being line-of-sight between the control device and the disinfection device.

In aspects, the controller may be configured to change the degree of control of the user interface element in controlling said operational parameter to a reduced degree of control when the line-of-sight condition comprises that no line-of sight is present between the control device and the disinfection device for a predetermined duration. Said predetermined duration may be for example at least 2 seconds, or at least 5 seconds, or at least 10 seconds. Additionally, or alternatively, said predetermined duration may be at most 10 seconds, or at most 1 minute. Other predetermined durations may be envisioned similarly. Hence, the line-of-sight is only broken when for the duration of said predetermined duration no line-of sight is present between the control device and the disinfection device. This may be advantageous, because the line-of-sight condition is not immediately determined for cases when a user stands in between the control device and the disinfection device, e.g. when walking away therefrom, but determined if said no line-of-sights continues for such a predetermined duration.

In an embodiment, the control device may comprise a transmitter for transmitting a control signal to the ultraviolet light-based disinfection device for controlling said operational parameter, wherein the controller is configured to control the transmitter to output said control signal based on a user selection of said user interface element. Said control signal may comprise, or may be indicative of, the operational parameter to be controlled and/or an associated control value to which said operational parameter is to be controlled (or set).

In an embodiment, the operational parameter of the ultraviolet light-based disinfection may comprise at least one of: an on/off condition; a spectral distribution of light; a type of ultraviolet light; a light intensity; a duration of UV-light emittance; a beam width; a beam direction. Such operational parameters of the ultraviolet light-based disinfection device may be relevant for reasons of safety.

Said ultraviolet light may comprise at least one of: Ultra Deep UV light in the range of 100-190 nm, Deep UV light in the range of 190-220 nm, UV-C light in the range of 220-280 nm, UV-B light in the range of 280-315 nm, UV-A light in the range of 315-400 nm, UV light at 254 nm. Such spectral distributions are known for their germicidal effect, of which at least part may negatively affect a person upon exposure to undesired doses and/or durations.

The user interface may be a digital user interface, such as a touchscreen window of a portable (smart) device. In an embodiment, the user interface element may comprise a button, a rotary knob, or a slider. Said user interface element may further be a switch, a menu, a drop-down list, a button array, a color wheel, or a selectable color space, or an option switch. Said button or slider may for example be a rendered touchscreen button or slider. Said button or slider may for example control the operational parameter of the UV-based disinfection device to a degree of control.

The user interface may alternatively be an analogue or physical user interface, such as a remote control device comprising the user interface element, wherein the user interface element may e.g. comprise a button, a rotary knob, a switch, or a slider. Said user interface element may thereby be mechanically controlled.

In aspects, the user interface element may comprise a voice input element for receiving a voice control input, wherein the voice control input is arranged for controlling said operational parameter of the ultraviolet light-based disinfection device to the degree of control. Changing the degree of control may thereby comprise limiting the voice input element in processing the received voice control input(s).

In an embodiment, the control device may be one of, or be part of one of: a wearable device, a smartwatch, a smartphone, smart glasses, an AR Headset, a tablet, a remote control, a switch, a laptop. In an embodiment, the user interface may be a touchscreen display.

Hence, in aspects, the user interface may be a touchscreen display, wherein the user interface element comprises a button or a slider, wherein said button or said slider is rendered, when enabled, on the touchscreen display; wherein the controller is configured to change the degree of control of the user interface element in controlling said operational parameter based on said spatial relation.

In aspects, said user interface element may be a plurality of user interface elements, wherein each respective user interface element of the plurality of user interface elements may control a corresponding (or respective) operational parameter of the ultraviolet light-based disinfection device. The controller may be configured to change the degree of control of at least one of the user interface elements of the plurality of user interface elements based on said spatial relation according to the invention. In aspects thereof, each user interface element of the plurality of user interface elements may be configured to control a different operational parameter of the UV light-based disinfection device. Moreover, the controller may be configured to change the number of enabled user interface elements of the plurality of user interface elements based on said spatial relation according to the invention. For example, the controller may be configured to gradually reduce the number of enabled user interface elements of the plurality of user interface elements in controlling their respective operational parameter when the distance of the control device relative to the disinfection location increases. Examples and embodiments provided above relating to the user interface element may mutatis mutandis apply for the aspect in which the user interface element is a plurality of user interface elements.

In aspects, the reduced degree of control may comprise the user interface element providing an indication message. Said indication message may be a visual and/or audio cue. For example, said indication message may be provided upon touch but not selection, or upon selection. For example, the user interface element may upon touch or selection provide a visual and/or audio cue, which visual and/or audio cue may induce a user to refrain from controlling said operational parameter of the UV light-based disinfection device to a certain, or full degree.

In aspects, the ultraviolet light-based disinfection device may be a lighting device, or be comprised in a lighting device. Hence, the lighting device may comprise the ultraviolet light-based disinfection device. The lighting device may e.g. be an ultraviolet light-based disinfection luminaire, such as an upper air disinfection luminaire, or a table-luminaire with disinfection properties.

In aspects, the controller may be configured to output a location indication via said user interface. The location indication may be, or may be indicative of, the location of the control device relative to the disinfection device and/or disinfection location. This may allow the control device to provide the user thereof to know the actual location of the control device relative to the ultraviolet light-based disinfection device and/or the disinfection location.

Furthermore, the present invention may define said spatial relation differently. Namely, in an aspect, the spatial relationship may be a location classification. Said location classification may be the classification of the location in which the ultraviolet light-based disinfection device and/or the control device is present. For example, the location classification may be a classification of rooms. The ultraviolet light-based disinfection device may be located in a first room, and the control device may be located in a second room. The controller of the control device may then be configured to change the degree of the control of the user interface element in controlling said operational parameter based on said classification of the room. For example, the degree of control of the user interface element in controlling said operational parameter may be different for when the control device is in a corridor, and/or in a remote different room, compared to a room in which the disinfection is performed. The controller may obtain, either receive or retrieve, a location classification from an external device, such as a presence sensor or localization system.

It is further an object of the invention to provide an improved system, which at least alleviates the problems and disadvantages mentioned above. Thereto, the invention provides a system comprising an ultraviolet light-based disinfection device arranged at a disinfection location and a control device according to the invention, wherein control device is arranged to control said ultraviolet light-based disinfection device. Moreover, the control device may be configured to convey a control signal to the ultraviolet light-based disinfection device, and the ultraviolet light-based disinfection device may be configured to obtain, receive or retrieve a control signal configured to control said operational parameter from the control device. In an embodiment, the UV light-based disinfection device may comprise a receiver for receiving said control signal. In an embodiment, the control device may comprise a transmitter for conveying said control signal to the ultraviolet light-based disinfection device, wherein the controller of the control device is configured to output said control signal based on a user selection of the user interface element. Said control signal may comprise, or may be indicative of, the operational parameter to be controlled and/or an associated control value to which said operational parameter is to be controlled (or set).

Thereby, advantages and/or embodiments applying to the control device according to the invention may mutatis mutandis apply to said system according to the invention.

It is further an object of the invention to provide an improved method of controlling a ultraviolet light-based disinfection device arranged at a disinfection location with a control device. Thereto, the invention provides a method of controlling a ultraviolet light-based disinfection device arranged at a disinfection location with a control device, wherein the control device is a portable device; wherein the control device comprises an user interface comprising a user interface element, wherein the user interface element is configured to control an operational parameter of the ultraviolet light-based disinfection device by providing a degree of control in controlling said operational parameter; wherein the method comprises: (i) determining a spatial relation of the control device relative to the ultraviolet light-based disinfection device and/or the disinfection location, and to (ii) changing the degree of control of the user interface element in controlling said operational parameter based on said spatial relation; wherein the spatial relation is a distance of the control device relative to the disinfection location. Thereby, advantages and/or embodiments applying to the control device according to the invention may mutatis mutandis apply to said method according to the invention.

For example, in an embodiment, the spatial relation may be a distance of the control device relative to the disinfection location, wherein the step of changing may comprise:
gradually reducing the degree of control of the user interface element in controlling said operational parameter when the distance of the control device relative to the disinfection location increases. Said ‘gradually reducing’ may also be ‘reducing’.

In an embodiment, the spatial relation is a distance of the control device relative to the disinfection location, wherein the step of changing may comprise changing the degree of control of the user interface element in controlling said operational parameter to a reduced degree of control when the distance of the control device relative to the disinfection location is above a first threshold distance and/or below a second threshold distance.

The invention further relates to a computer program product. Hence, the invention provides a computer program product for a computing device, the computer program product comprising computer program code to perform a method according to the invention when the computer program product is run on a processing unit of the computing device.

Thus, aspects of the invention may be implemented in a computer program product, which may be a collection of computer program instructions stored on a computer readable storage device which may be executed by a computer. The instructions of the present invention may be in any interpretable or executable code mechanism, including but not limited to scripts, interpretable programs, dynamic link libraries (DLLs) or Java classes. The instructions can be provided as complete executable programs, partial executable programs, as modifications to existing programs (e.g. updates) or extensions for existing programs (e.g. plugins). Moreover, parts of the processing of the present invention may be distributed over multiple computers or processors.

Throughout the application, in alternative phrasing, the user interface element may be configured to set a value of an operational parameter of the ultraviolet light-based disinfection device and change a degree of control in setting said operational parameter, wherein the controller is configured to change the degree of control of the user interface element in setting said operational parameter based on said spatial relation. Hence, in an aspect, the invention may provide, a control device for controlling an ultraviolet light-based disinfection device arranged at a disinfection location, wherein the control device comprises: a user interface comprising a user interface element, wherein the user interface element is configured to set a value of an operational parameter of the ultraviolet light-based disinfection device and to change a degree of control in setting said operational parameter; a controller configured to: (i) determine a spatial relation of the control device relative to the ultraviolet light-based disinfection device and/or the disinfection location, and to (ii) change the degree of control of the user interface element in setting a value of said operational parameter based on said spatial relation.

In aspects, said controller may be a local controller, which operates in conjunction with a master controller, wherein the master controller is external to the control device. Hence, the master controller may be configured to control the controller to perform the tasks of the controller as mentioned in the present application.

In aspects of the invention, the ultraviolet light-based disinfection device may be an ionizer. Hence, the invention may provide, a control device for controlling an ionizer arranged at a disinfection location, wherein the control device comprises: a user interface comprising a user interface element, wherein the user interface element is configured to control an operational parameter of the ionizer by providing a degree of control in controlling said operational parameter; a controller configured to: (i) determine a spatial relation of the control device relative to the ionizer and/or the disinfection location, and to (ii) change the degree of control of the user interface element in controlling said operational parameter based on said spatial relation. The operational parameter may comprise one of: an ionization duration, an ionization voltage, an ionization intensity, a type of ionized molecules. Thereby, advantages and/or embodiments applying to the control device and system according to the invention may mutatis mutandis apply to said aspect according to the invention in which the control device is configured to control an ionizer.

### BRIEF DESCRIPTION OF THE DRAWINGS

The invention will now be further elucidated by means of the schematic non-limiting drawings:

FIG. 1 depicts schematically an embodiment of a system comprising a control device and an ultraviolet light-based disinfection device according to the invention;

FIG. 2 depicts schematically an operation of the control device of the embodiment depicted in FIG. 1;

FIG. 3 depicts schematically an operation of the control device of the embodiment depicted in FIG. 1;

FIG. 4 depicts schematically an operation of the control device of the embodiment depicted in FIG. 1;

FIG. 5 depicts schematically a user interface according to the invention;

FIG. 6 depicts schematically an embodiment of a system comprising a control device and an ultraviolet light-based disinfection device according to the invention;

FIG. 7 depicts schematically a method according to the invention.

### DETAILED DESCRIPTION OF THE EMBODIMENTS

Ultraviolet (UV) light-based disinfection devices require improved safety measures. Ultraviolet light may namely be dangerous to people and/or animals, even though it provides a germicidal effect and is very useful for disinfection. As such ultraviolet light-based disinfection device may be remotely controlled, it may be dangerous to allow a user, which is too remote to the disinfection device and/or the associated disinfection location, control of the ultraviolet light-based disinfection device, because such a remote user may not oversee the disinfection device and/or the associated disinfection location. Similarly, it may be dangerous to enable control of an ultraviolet disinfection device, when a user is too close by to the ultraviolet light-based disinfection device and/or the associated disinfection location.

All in all, a clear need exists to improve the safety of UV light-based disinfection devices, in particular for domestic (non-professional) users. It would also be more convenient to control the UV light-based disinfection device remotely, with improved safety.

The present invention addresses this problem by proposing a control device, wherein a user interface element controlling an operational parameter of an ultraviolet light-based disinfection device to a degree of control is changed in the degree of control based on a spatial relation of the control device relative to the disinfection device and/or disinfection location.

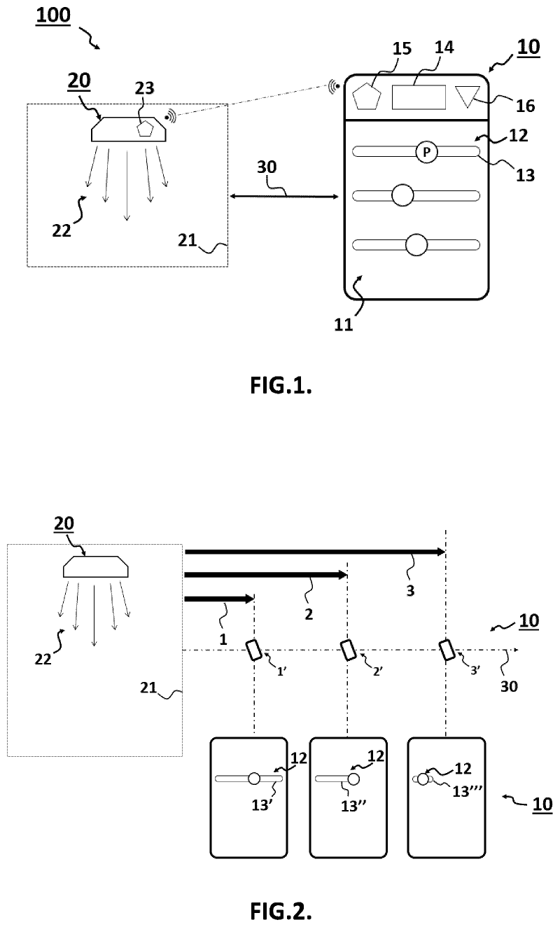

FIG. 1 depicts schematically, by non-limiting example, an embodiment of a system 100 according to the invention. The system 100 comprises a control device 10 according to the invention and an ultraviolet light-based disinfection device 20 according to the invention. The control device 10 is configured to control the ultraviolet light-based disinfection device 20.

The control device 10 is a portable device. For example, a smartphone. Alternatively, the control device may be one of, or be part of one of: a wearable device, a smartwatch, a smartphone, smart glasses, an AR Headset, a tablet, a remote control, a switch, a laptop.

The ultraviolet light-based disinfection device 20 is arranged at a disinfection location 21. Here, the disinfection location is a room. The ultraviolet light-based disinfection device is, in operation, disinfecting said room. The ultraviolet light-based disinfection device may for example be a lighting device or luminaire, such as a table lamp, ceiling luminaire, or upper-air disinfection luminaire. Alternatively, said disinfection location may be any other space or enclosure, which is to be disinfected by the ultraviolet light-based disinfection device.

The ultraviolet light-based disinfection device 20 further comprises a light source (not explicitly depicted) for emitting ultraviolet light 22. The ultraviolet light 22 is operated according to an operational parameter of the ultraviolet light-based disinfection device 20. Here, the operational parameter is ultraviolet light intensity, but alternatively the operational parameter of the ultraviolet light-based disinfection device may comprise one of: an on/off condition; a spectral distribution of light; a type of ultraviolet light; a light intensity; a duration of UV-light emittance; a beam width; a beam direction. The ultraviolet light-based disinfection device may further optionally comprise optics for managing/controlling the ultraviolet light emitted by the light source. Said optics may e.g. be lenses, diffractors, reflectors, light guides, etc.

The ultraviolet light-based disinfection device 20 further comprises a receiver 23 (or: transceiver) for receiving a control signal to operate the ultraviolet light-based disinfection device 20 according to said operational parameter. Said control signal is conveyed to the ultraviolet light-based disinfection device 20 from the control device 10, which conveying may comprise the control device 10 either directly transmitting said control signal or conveying said control signal via an intermediate device or node, such as a server or router. Here, the transceiver 23 is a wireless receiver, but it may alternatively be a receiver operating via a wired communication modality. The wireless receiver may e.g. operate according to the wireless modality of: Bluetooth, Wi-Fi, RF, ZigBee, Lo-Ra, Li-Fi, VLC, 4G, 5G and/or UWB.

Referring to FIG. 1, the control device 10 comprises a transmitter 15 for conveying a control signal to the ultraviolet light-based disinfection device 20. Said transmitter may alternatively be a transceiver, thereby also having a receiving capability. Such a transceiver may thus contain a transmitter and a receiver. The transmitter 15 is a radiofrequency transmitter, but may alternatively operate with the mentioned wireless modalities of: Bluetooth, Wi-Fi, RF, ZigBee, Lo-Ra, Li-Fi, VLC, 4G, 5G and/or UWB.

The control device 10 further comprises a user interface 11 and a controller 14. Here, the user interface 11 is a touchscreen display, but may alternatively be an analogue, physical, or mechanical, user interface. The user interface 11 comprises a user interface element 12. The user interface element 12 controls an operational parameter of the ultraviolet light-based disinfection device 20 by providing a degree of control in controlling said operational parameter. Alternatively phrased, said user interface element 12 may be configured to set a value of the operational parameter of the ultraviolet light-based disinfection device 20 and to change a degree of control in setting said operational parameter.

In the present embodiment, the operational parameter, as mentioned, is ultraviolet light intensity. In the present embodiment, the user interface element 12 is a slider. The user interface element provides a degree of control 13, namely a range (or: set) of intensity values that is selectable by the slider 12. This range (or: set) may initially be 0% to 100%. Hence, the degree of control 13 may be 0% to 100%.

Alternatively, said user interface element may comprise one of: a button, a rotary knob, a switch, a menu, a drop-down list, a button array, a color wheel, or a selectable color space, or an option switch. For example, the user interface may be an analogue or physical user interface with an array of buttons, and the user interface element may be the array of buttons (e.g. a plus and minus button to respectively increase and decrease light intensity), or a single button of say array of buttons.

Moreover, the control device 10 comprises the transmitter 15. The transmitter 15 conveys a control signal to the ultraviolet light-based disinfection device 20 for controlling said operational parameter. Here, said conveying is transmitting the control signal to the ultraviolet light-based disinfection device 20. The controller 14 is configured to control the transmitter 15 to output, i.e. transmit, said control signal based on a user selection of said user interface element 12, namely the selection being to a degree of control 13.

Still referring to FIG. 1, the controller 14 is configured to determine a spatial relation of the control device 10 relative to the ultraviolet light-based disinfection device 20 and/or the disinfection location 21. In the present embodiment, said spatial relation is distance 30 of the control device 10 relative to the disinfection location 21, though it may also be relative to the ultraviolet light-based disinfection device 20. This distance 30 may be defined as the shortest distance between the control device 10 and the disinfection location 21. Alternatively, said spatial relation may for example be a line-of-sight condition or a location classification (such as room type or room ID).

The control device 10 may determine said spatial relation, i.e. said distance 10, as follows. In the present embodiment, the control device 10 comprises a receiver 16. The receiver 16 and the transmitter 15 may in examples be embodied in a single transceiver. Hence, in alternative aspects, the control device may comprise a transceiver comprising said transmitter and said receiver. The receiver 16 is arranged for receiving a wireless signal, wherein the wireless signal comprises an indicator indicative of the distance 30 of the control device 10 relative to the disinfection location 21. The wireless signal is thereby a radiofrequency signal. The controller 14 is then configured to determine said distance of the control device 10 relative to the disinfection location 21 based on said indicator. Here, the indicator comprised in the wireless signal is distance data. Moreover, the control device 10 receives said distance data from a further device, which is here a localization system, that tracks and knows the location of the control device 10 relative to the disinfection location 21. For example, the localization system may be a positioning system, an asset tracking system, etc. In embodiments, not depicted, the system according to the invention may include such a further device, as said localization system.

Alternative options and/or embodiments may similarly be envisioned. For example, the control device 10 may comprise a location determination unit to determine the indicator itself, which indicator is being used to determine the distance of the control device relative to the disinfection location. The location determination unit may e.g. be a GPS antenna, which determines a GPS location of the control device and compares this to a predefined location of the ultraviolet light-based disinfection device and/or the disinfection location. Other localization modalities, i.e. other than GPS, such as Wi-Fi, VLC, and/or BLE enabled localization, may similarly be envisioned.

The location determination unit may also be a radiofrequency transceiver, or receiver, so as to determine the distance of the control device relative to the disinfection device via signal strength measurements. Said signal strength may be RSSI. Hence, the indicator may be signal strength. Said radiofrequency transceiver may yet alternatively receive location information from another device, such as a positioning system or presence sensor, such that the controller may compute said distance from this location information.

Yet alternatively, said receiver may be a Li-Fi receiver, a VLC receiver, an IR receiver, a camera, or a microphone.

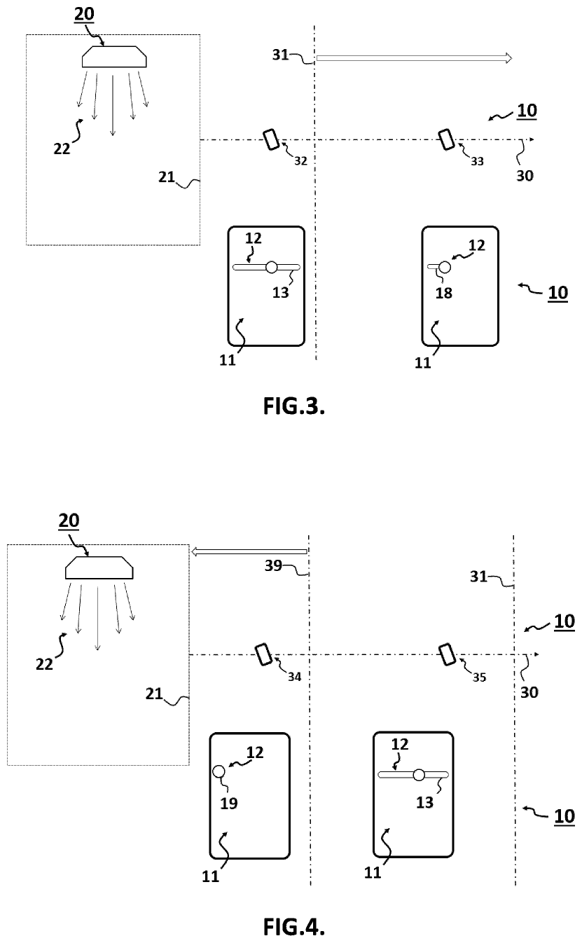

Still referring to FIG. 1, the controller 14 is configured to change the degree of control 13 of the user interface element 12 in controlling said operational parameter based on said spatial relation. Namely, the controller changes the degree of control 13, i.e. the range (or: set) of intensity values that is selectable by the slider 12, based on the determined spatial relation. This changing function is explained in more detail according to one example in FIG. 2 and according to another example in FIG. 3. An alternative, or additional, exemplar operation is provided in FIG. 4.

FIG. 2 depicts schematically, by non-limiting example, an operation of the control device 10 of the embodiment depicted in FIG. 1 according to the invention. Namely, in this exemplar embodiment and operation, in terms of distance of the control device 10 relative to the disinfection location 21, the control device 10 is being moved further away from the disinfection location 21. More specifically, the control device 10 is moved from a first location 1′ at a first distance 1, to a further second location 2′ at a second distance 2, to a further third location 3′ at a third distance 3 relative to the disinfection location 21. Hence, the distance 30 of the control device 10 relative to the disinfection location 21 increases in this exemplar embodiment.

Still referring to FIG. 2, the controller 14 of the control device 10 is gradually reducing the degree of control 13 of the user interface element 12 in controlling said operational parameter when the distance 30 of the control device 10 relative to the disinfection location 21 increases. Namely, as depicted, at the first distance 1 and/or first location 1′, the degree of control 13′ of the user interface element 12 in controlling said operational parameter is the full range of 0% to 100% ultraviolet light intensity, which is selectable by the slider 12. Namely, as depicted, at the second distance 2 and/or second location 2′, the degree of control 13″ of the user interface element 12 in controlling said operational parameter is a limited range of 0% to 60% ultraviolet light intensity, which is selectable by the slider 12. Namely, as depicted, at the third distance 3 and/or third location 3′, the degree of control 13′″ of the user interface element 12 in controlling said operational parameter is a limited range of 0% to 10% ultraviolet light intensity, which is selectable by the slider 12.

Even further, in further aspects, at an even further fourth distance (not depicted), the degree of control may be zero. This may mean that the user interface element does not provide any degree of control of the operational parameter at such a fourth distance from the disinfection location. The user interface element may for example be disabled (in controlling said operational parameter of the ultraviolet light-based disinfection device).

Hence, alternatively phrased, the user interface element may be configured to set the value of the operational parameter and change a degree of control in setting said operational parameter, i.e. e.g. configured to set a value between 0-100% as degree of control and change this degree of control to a value range to e.g. 0-60%.

As a result, referring to FIG. 2, the controller 14 of the control device 10 is configured to gradually reduce (or: decrease, or: reduce) the degree of control 13 of the user interface element 12 in controlling said operational parameter when the distance 30 of the control device 10 relative to the disinfection location 21 increases. Here, as partly mentioned, the reducing of said degree of control 13 comprises limiting the user interface element 12 to a limited set of values for controlling said operational parameter.

Alternatively, said reducing may comprise limiting the user interface element 12 to a limited set of options for controlling said operational parameter, and/or fully disabling the user interface element in controlling said operational parameter, and/or adapting a graphical representation of the user interface element in controlling said operational parameter. Said disabling may also comprise disabling the processing of a voice control user input, when said user interface element is a voice control input element. Said adapting the graphical representation may, in case of a GUI be for example: greying out a graphical representation of the user interface element, or rendering a strikethrough, or adapting the dimensions of the user interface element, e.g. making the user interface element smaller while distance between the control device and the disinfection location and/or said disinfection device increases. Yet alternatively, said reduced degree of control may comprise fully disabling the user interface element in controlling said operational parameter. Yet alternatively, said reduced degree of control may comprise adapting a graphical representation of the user interface element in controlling said operational parameter.

FIG. 3 depicts schematically, by non-limiting example, an operation of the control device 10 of the embodiment depicted in FIG. 1 according to the invention. Namely, in this exemplar embodiment and operation, the control device 10 may be located at various locations 32, 33 in space, wherein each respective location is at a certain distance 30 relative to the disinfection location 21.

Still referring to FIG. 3, the controller 14 of the control device 10 is configured to change the degree of control 13 of the user interface element 12 in controlling said operational parameter to a reduced degree of control 18 when the distance 30 of the control device 10 relative to the disinfection location 21 is above a first threshold distance 31.

As partly mentioned before, a user may control the operational parameter of the ultraviolet light-based disinfection device 20 by manipulating the user interface element 12 of the control device 10, wherein the user interface element 12 provides a certain degree of control 13, 18 for said manipulation, and in thereby controlling said operational parameter. However, said degree of control 13 is changed to a reduced degree of control 18 when the distance 30 of the control device 10 relative to the disinfection location 21 is above the first threshold distance 31. Here, the first threshold distance is ten meter, but may alternatively be any other value, such as e.g. one meter, two meter, five meter, hundred meter, one kilometer, at least ten meter. Here, the first threshold distance is predefined and stored in the controller, but may alternatively be obtained from another source. For example, the controller may obtain said threshold distance from a remote server, or e.g. the ultraviolet light-based disinfection device itself. The first threshold distance may alternatively also be determined based on the operational parameter to control. Hence, operational parameters that may classify as riskier when controlled (for example the intensity of ultraviolet (e.g. UV-C) light comprises much risk to humans and animals when controlled), may provide a lower first threshold distance than operational parameters that are classified relatively as less risky (for example, the beam width).

More specifically, still referring to FIG. 3, as the user interface element is a slider 12, which is rendered on the user interface 11, the degree of control 13, 18 is a range (or: set) of intensity values that is selectable by the slider 12. When the control device 10 is at a first location 32, which is below the first threshold distance 31, the controller 14 is configured to control the user interface 11 to render the user interface element 12 providing the degree of control 13. Here, the degree of control 13 is a selectable range of intensity values between 0% and 100% with the slider 12. When the control device 10 is at a second location 33, which is above the first threshold distance 31, the controller 14 is configured to control the user interface 11 to render the user interface element 12 providing the reduced degree of control 18. Here, the reduced degree of control 18 is a selectable range of intensity values between 0% and 20% with the slider 12. Other value ranges of the degree of control and the reduced degree of control may be envisioned similarly.

Alternatively, said user interface element may comprise at least one of: a button, a switch, a menu, a rotary knob, a drop-down list, a color wheel, a button array, an option switch, a switch, a voice input element. Alternatively, said reduced degree of control may comprise limiting the user interface element to a limited set of options for controlling said operational parameter. For example, provided that the user interface element providing the degree of control is an array of three buttons, respectively indicative of controlling the operational parameter of UV-A, UV-B, UV-C radiation of the ultraviolet light-based disinfection device, the reduced degree of control may only be a single button with e.g. UV-A radiation. Such UV-A radiation may be less harmful to people and animals compare to UV-C. Hence, according to the invention, the reduced degree of control, may be proportionally reduced in relation to the risk or danger of the operational parameter to be controlled with the user interface element.

Still referring to the embodiment and options depicted in FIGS. 1, 2 and 3. It may be undesired and a safety risk to allow a user to control the control device 10 when the control device 10 is too far away from disinfection location 21, and/or the ultraviolet light-based disinfection device 20, because such a user may control a dangerous disinfection device 20 without being near said disinfection device 20 and without being able to assess or monitor the corresponding disinfection location 21.

Therefore, since the degree of control 13 of the user interface element 12 in controlling the operational parameter of the ultraviolet light-based disinfection device 20 is made dependent on said spatial relation of distance, the control device according to the present invention may dynamically change the degree of control 13 of the ultraviolet light-based disinfection device 20, when the control device 10 is too far away from the UV-disinfection device 20 and/or disinfection location 21. This renders an improved safety measure, or advantageously introduces a safety constraint to the control device 10. Hence, the embodiment and options depicted in FIGS. 1, 2 and 3 are advantageous.

However, on the other hand, it may be a safety risk to allow a user to control a control device, for example the control device 10 as depicted in the embodiment of FIGS. 1-3, when such a control device 10 is too close to the ultraviolet light-based disinfection device 20 and/or disinfection location 21. Therefore, this problem may additionally or alternatively be addressed by a control device according to the invention.

FIG. 4 depicts schematically, by non-limiting example, an additional or alternative operation of the control device 10 of the embodiment depicted in FIGS. 1 to 3 according to the invention. Namely, in this exemplar embodiment and operation, the control device 10 may be located at various locations 34, 35 in space, wherein each respective location is at a certain distance 30 relative to the disinfection location 21.

Still referring to FIG. 4, the controller (not depicted) of the control device 10 is configured to change the degree of control 13 of the user interface element 12 in controlling said operational parameter to a reduced degree of control 19 when the distance 30 of the control device 10 relative to the disinfection location 21 is below a second threshold distance 39. This operation of the controller may be an alternative to the operation of the controller of the embodiments and options depicted in FIGS. 1-3, but may also be in addition thereto.

As partly mentioned before, a user may control the operational parameter of the ultraviolet light-based disinfection device 20 by manipulating the user interface element 12 of the control device 10, wherein the user interface element 12 provides a certain degree of control 13, 19 for said manipulation, and in thereby controlling said operational parameter. However, said degree of control 13 is changed to a reduced degree of control 19 when the distance 30 of the control device 10 relative to the disinfection location 21 is below the second threshold distance 39. The second threshold distance may for example be one of: one meter, five meter, ten meter.

More specifically, as the user interface element is a slider 12, which is rendered on the user interface 11, the degree of control 13, 19 is a range (or: set) of intensity values that is selectable by the slider 12. When the control device 10 is at a first location 35, which is above the second threshold distance 39, the controller 14 is configured to control the user interface 11 to render the user interface element 12 providing the degree of control 13. Here, the degree of control 13 is a selectable range of intensity values between 0% and 100% with the slider 12. When the control device 10 is at a particular location 34, which is below the second threshold distance 39, the controller 14 is configured to control the user interface 11 to render the user interface element 12 providing the reduced degree of control 19. Here, the reduced degree of control 18 is fully disabling the slider 12, or only allowing the 0% intensity value to be selectable by the slider 12.

Alternatively, the controller may also adapt a graphical representation of said slider at the reduced degree of control. For example, the slider may be greyed-out, such that a user is prompted not to use said slider, as the visibility of the slider is made different or more difficult. Similarly, the controller may adapt a size of the graphical representation of the slider.

All in all, referring to FIG. 4, as less control of the ultraviolet light-based disinfection device 20 is allowed when the control device 10, and associated user thereof, is closer to the disinfection location 21, and more when further away from the disinfection location 21, it is be prevented that a too close-by user may cause a risk of accidents or dangerous situations at the disinfection location 21 and hence to the user itself.

Referring to FIG. 3 and FIG. 4, in embodiments, the second threshold distance 39 may be smaller than the first threshold distance 31, i.e. the second threshold distance is (located) closer to the disinfection location than the first threshold distance 31. Here, the first threshold distance 31 and/or the second threshold distance 39 are predefined. For example, the first threshold distance 31 and/or the second threshold distance 39 may be stored in the controller 14 or a memory associated with the controller, such that the controller 14 may determine the first threshold distance 31 and/or the second threshold distance 19 without needing to receive or retrieve the first threshold distance 31 and/or the second threshold distance 39 from another source. Alternatively, the controller may obtain, receive, or retrieve said first threshold distance and/or said second threshold distance from another source, such as a further device, or a Building Information Model, which may e.g. store commissioning information where the UV light-based disinfection device is installed, and which safety conditions apply (i.e. the first threshold distance and/or the second threshold distance).

In alternative embodiments, not depicted, the control device of the embodiments depicted in FIG. 1-3 may be a control system comprising the controller and the user interface, wherein the controller may be separate, e.g. remote from the user interface.

At least partly similar to the embodiments depicted in FIGS. 1-4, but in an alternative embodiment, the user interface element may be an array of buttons. FIG. 5 depicts schematically, by non-limiting example, said alternative user interface 61 according to the invention, which user interface 61 is comprising a user interface element 62 configured to control an operational parameter of an ultraviolet light-based disinfection device arranged at a disinfection location by providing a degree of control 63 in controlling said operational parameter. Here, the operational parameter is a type of ultraviolet light. The user interface 61 is thereby comprised by a control device 60. The control device 60 further comprises a controller 64.

The array of buttons thereby control the type of ultraviolet light provided by the ultraviolet light-based disinfection device. The array of buttons comprises a first button 621 controlling the ultraviolet light-based disinfection device to emit UV-A light. The array of buttons comprises a second button 622 controlling the ultraviolet light-based disinfection device to emit UV-B light. The array of buttons comprises a second button 623 controlling the ultraviolet light-based disinfection device to emit UV-C light. The controller 64 is configured to determine a spatial relation of the control device 60 relative to said ultraviolet light-based disinfection device and/or said disinfection location. The controller is further configured to change the degree of control 63 of the user interface element in controlling said operational parameter based on said spatial relation. The spatial relation may e.g. be distance, or line-of-sight. The degree of control 63 is the option to select UV-A, UV-B, or UV-C with said button array 62. In further embodiments, the controller 64 changes the degree of control 63 of the user interface element 62 in controlling said operational parameter to a reduced degree of control 66 when the distance of the control device 60 relative to the disinfection location and/or disinfection device is above a first threshold distance and/or below a second threshold distance.

Here, the reduced degree of control 66 comprises limiting the set of options for controlling the operational parameter of the type of ultraviolet light to be provided by the ultraviolet light-based disinfection device. Namely, when the controller determines that the distance of the control device 10 relative to the disinfection location and/or disinfection device is above a first threshold distance and/or below a second threshold distance, the button array will only render the option of UV-A light. Hence, the control device can only control the ultraviolet light-based disinfection device to emit UV-A light by user selection of the user interface element 62.

Alternatively, said reduced degree of control may comprise adapting a graphical representation of the user interface element in controlling said operational parameter. For example, a graphical representation of the user interface element may be made transparent, or greyed-out, such that the user interface element becomes less visible and noticeable for interfacing with it. For example, a graphical representation of the user interface element may comprise a strikethrough, indicative that said user interface element is not to be used. Similarly, for example, a graphical representation of the user interface element may comprise an alerting feature, indicative that said user interface element is not to be used. Yet alternatively, said reduced degree of control may comprise adapting a size of the user interface element, for example making the size of the user interface element smaller in the reduced degree of control.

Alternatively, said user interface element may be a voice input means for receiving voice control commands to control said operational parameter. For example, the voice input means may be a microphone. The voice control commands, which are receivable and processable by the voice input means and the controller of the control device at the degree of control, may mutatis mutandis to the button array mentioned above comprise “turn to UV-A”, “turn to UV-B” and/or “turn to UV-C”. The reduced degree of control may then comprise reducing the possible voice control commands, mutatis mutandis to the button array mentioned above, to only the voice control command “turn to UV-A”.

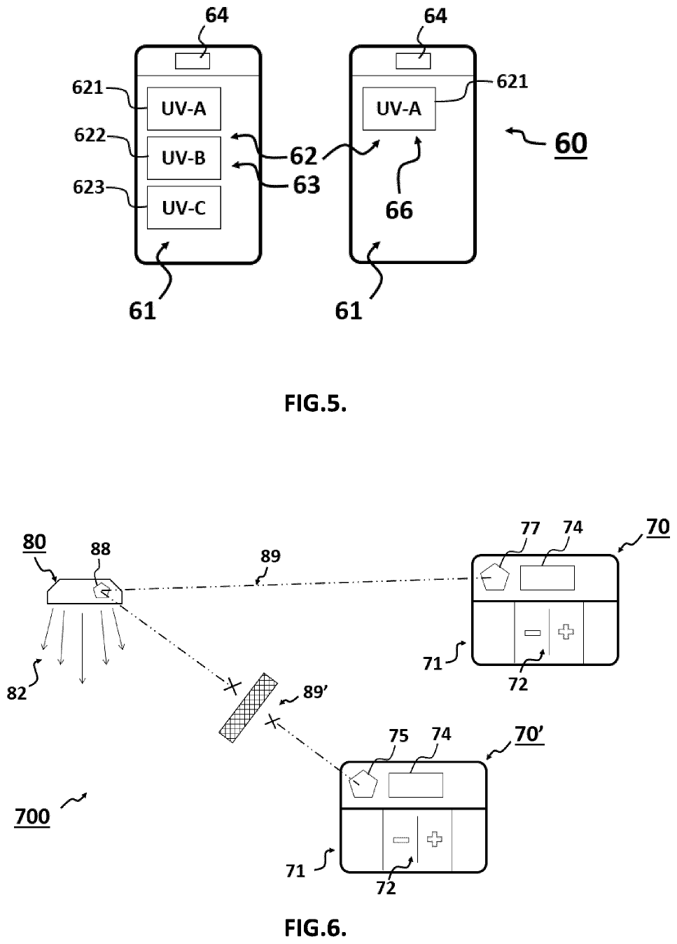

FIG. 6 depicts schematically, by non-limiting example, an embodiment of a system 700 according to the invention. The system 700 comprises an ultraviolet light-based disinfection device 80 arranged at a disinfection location 81. The ultraviolet light-based disinfection device 80 disinfects, in operation, said disinfection location 81 with ultraviolet light 82 having a lighting characteristic. The ultraviolet light-based disinfection device 80 will then be operated at an operational parameter causing said lighting characteristic of the ultraviolet light 82.

Here, the ultraviolet light-based disinfection device 80 is a mobile device, which may move from the current disinfection location 81 to another disinfection location in the future. The ultraviolet light-based disinfection device 80 further comprises a light transmitter for transmitting a light signal in operation. Here, the light signal is infrared, but may alternatively be visible light. Said light transmitter may alternatively be a radiofrequency transmitter for emitting a radiofrequency signal, or ultrasound emitter for emitting an ultrasound signal. The light signal may thereby be coded for improved safety purposes. The light signal may comprise an identifier of the ultraviolet light-based disinfection device. Said light signal may alternatively be encrypted. The light transmitter may either be housed in a same housing as the light sources of the ultraviolet light-based disinfection device, but may also be separate and standalone, i.e. in a separate transmitter unit associated with the other components of the ultraviolet light-based disinfection device.

The system 700 further comprises a control device 70. The control device 70 is configured to control the ultraviolet light-based disinfection device 80. Namely, the control device 70 comprises a wireless communication module to convey a control signal to the ultraviolet light-based disinfection device 80, so as to control an operational parameter of the ultraviolet light-based disinfection device 80 with said control signal. The ultraviolet light-based disinfection device 80 may thereby comprise a communication module for receiving said control signal, or another signal indicative of, or comprising, said control signal. The wireless communication module is a transmitter, which transmits the control signal via a wireless communication modality. This wireless communication modality is Bluetooth, but may alternatively be any other wireless communication modality as mentioned in the present application.

Referring to FIG. 6, the control device 70 comprises a user interface 71, a light receiver 77, and a controller 74. The user interface 71 comprises a user interface element 72. The user interface 71 is an analogue user interface, which contains an array of mechanical switches. The user interface element is a binary switch 72 of said array of mechanical switches. For example, the control device comprising the user interface 71 may be a Philips Hue Switch. The binary switch 72 is configured to control an operational parameter of the ultraviolet light-based disinfection device by providing a degree of control in controlling said operational parameter. Here, the operational parameter is ultraviolet light intensity of the ultraviolet light 82. The degree of control is binary. Namely, either increase or decrease the ultraviolet light intensity with said binary switch 72.

The light receiver 77 of the control device 70 is arranged to receive the light signal of the ultraviolet light-based disinfection device 80. The controller 74 of the control device 70 is configured to determine a spatial relation of the control device 70 relative to the ultraviolet light-based disinfection device 80. Here, the spatial relation is a line-of-sight between the control device 70 and the ultraviolet light-based disinfection device 80.

Hence, the controller 74 is configured to a line-of-sight condition 89, 89′ between the control device 70 and the ultraviolet light-based disinfection device 80. This line-of-sight condition 89, 89′ is also binary. Namely: The line-of-sight condition 89 is determined to have line-of-sight present between the control device 70 and the ultraviolet light-based disinfection device 80 when the light receiver 77 is receiving the light signal of the ultraviolet light-based disinfection device 80. The line-of-sight condition 89′ is determined not to have line-of-sight present between the control device 70′ and the ultraviolet light-based disinfection device 80 when the light receiver 77 is not receiving (anymore) the light signal of the ultraviolet light-based disinfection device 80.

Moreover, still referring to FIG. 6, the controller 74 is configured to determine a line-of sight condition 89, 89′ between the control device and the disinfection device, and to change the degree of control of the binary switch 72 in controlling said operational parameter to a reduced degree of control when the line-of-sight condition 89′ comprises that no line-of sight is present between the control device 70′ and the ultraviolet light-based disinfection device 80.

Here, the reduced degree of control is the controller disabling the user interface element 12, i.e. the binary switch 12, in controlling said operational parameter. Hence, the controller will not respond to any user interaction with said binary switch 12 when the line-of-sight condition 89′ is that a line-of sight present between the control device 70 and the ultraviolet light-based disinfection device 80.

In alternative embodiments, said user interface element may be a voice input element, such as a microphone. The reduced degree of control may then comprise adapting the processing of a user (voice) input resulting from the voice input element, for example stopping processing said user (voice) input.

In alternative embodiments, vice versa, the light transmitter of the ultraviolet light-based disinfection device may be a light receiver, and the light receiver of the control device may be a light transmitter. The controller of the control device may then be in wireless communication with the ultraviolet light-based disinfection device, so as to determine said line-of-sight condition, i.e. whether the transmitted light signal is still able to be received.

Alternatively, said light transmitter and said light receiver may respectively be a radiofrequency transmitter and a radiofrequency receiver. In such an alternative embodiment, the line-of-sight condition may then comprise line-of-sight to be present when the radiofrequency signals are being able to be received by the radiofrequency receiver, and may then comprise the line-of-sight not to be present when the radiofrequency signals are not being received (anymore) by the radiofrequency receiver.

In an embodiment, not depicted, the invention provides the control device of the embodiments depicted in the FIGS. 1-6, but wherein the spatial relationship is not distance or line-of-sight, but a location classification. Namely, the controller of the control device may be configured to obtain a location classification signal.

The location classification signal may e.g. be obtained by the controller determining the location of the control device and matching said location to a location classification as stored in a local database, wherein the match renders said location classification signal. The location may thereby be determined via a location sensor in the control device itself, such as a GPS antenna or BLE antenna, or from a positioning/localization system providing said location to the control device wirelessly. For example, the location classification signal may be indicative of a certain space the control device is currently present in, which is the location classification. The location classification is for example a room, a corridor, a first room, a second room, an outdoor space, a disinfection location, etc. Thus, when the controller knows the location of the control device, said location classification signal and associated location classification can then also be determined.

Moreover, the location classification signal may in other examples be received by the control device from an external device. Said external device may e.g. be a sensor, a luminaire, an asset tracking system, a building management device, a room planning device, etc. The external device may thereby provide the control device, and the controller, with the location classification.

Upon determining the location classification, mutatis mutandis to the embodiments depicted in the FIGS. 1-6, the controller of the control device is configured to change the degree of control of the user interface element in controlling said operational parameter based on said location classification. For example, the degree of control may be a reduced degree of control according to the invention when the location classification is the disinfection location, and/or an outdoor space remote to the disinfection location, and/or a floor different to a floor associate with the disinfection location. For example, some rooms of a building may cause a reduced degree of control, compared to other rooms of said building.

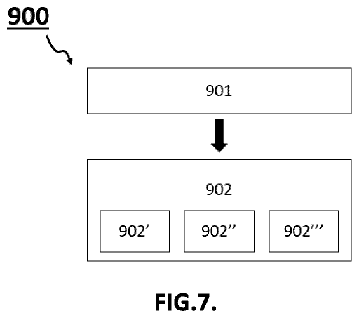

FIG. 7 depicts schematically, by non-limiting example, an embodiment of a method 900 according to the invention. The method 900 relates to controlling an ultraviolet light-based disinfection device arranged at a disinfection location with a control device. The control device may be a control device according to the embodiments depicted in FIGS. 1-6. The control device comprises a user interface element, wherein the user interface element is configured to control an operational parameter of the ultraviolet light-based disinfection device by providing a degree of control in controlling said operational parameter. The method 900 comprises the step 901 of determining a spatial relation of the control device relative to the ultraviolet light-based disinfection device and/or the disinfection location. The method 900 comprises the step 902 of changing the degree of control of the user interface element in controlling said operational parameter based on said spatial relation.

Thereby, said step 902 of changing the degree of control comprises changing the degree of control of the user interface element in controlling said operational parameter to a reduced degree of control when the distance of the control device relative to the disinfection location is above a first threshold distance and/or below a second threshold distance. Thereby, the second threshold distance is smaller than the first threshold distance, i.e. the second threshold distance is closer to the disinfection location than the first threshold distance.

Alternatively, or additionally, said step of changing may comprise to gradually reducing the degree of control of the user interface element in controlling said operational parameter when the distance of the control device relative to the disinfection location increases.

Moreover, said step 902 of changing the degree of control to a reduced degree of control may comprise a step 902′ of limiting the user interface element to a limited set of values or options for controlling said operational parameter. Moreover, said step 902 of changing the degree of control to a reduced degree of control may comprise a step 902″ of disabling the user interface element in controlling said operational parameter. Moreover, said step 902 of changing the degree of control to a reduced degree of control may comprise a step 902′″ of adapting a graphical representation of the user interface element in controlling said operational parameter.

1. A control device for controlling an ultraviolet light-based disinfection device arranged at a disinfection location, wherein the control device is a portable device, wherein the control device comprises:
a user interface comprising a user interface element, wherein the user interface element is configured to control an operational parameter of the ultraviolet light-based disinfection device by providing a degree of control in controlling said operational parameter;
a controller configured to:
(i) determine a spatial relation of the control device relative to the ultraviolet light-based disinfection device and/or the disinfection location, and to
(ii) change the degree of control of the user interface element in controlling said operational parameter based on said spatial relation;
wherein the spatial relation is a distance of the control device relative to the disinfection location.

2. The control device according to claim 1, wherein the controller is configured to reduce the degree of control of the user interface element in controlling said operational parameter when the distance of the control device relative to the disinfection location increases.

3. The control device according to claim 1, wherein the controller is configured to change the degree of control of the user interface element in controlling said operational parameter to a reduced degree of control when the distance of the control device relative to the disinfection location is above a first threshold distance and/or below a second threshold distance.

4. The control device according to claim 3, wherein the second threshold distance is smaller than the first threshold distance.

5. The control device according to claim 3, wherein the reduced degree of control comprises limiting the user interface element to a limited set of values or options for controlling said operational parameter.

6. The control device according to claim 3, wherein the reduced degree of control comprises fully disabling the user interface element in controlling said operational parameter.

7. The control device according to claim 3, wherein the reduced degree of control comprises adapting a graphical representation of the user interface element in controlling said operational parameter.

8. The control device according to claim 3, wherein the reduced degree of control comprises adapting a size of the user interface element.

9. The control device according to claim 1, wherein the controller comprises a receiver for receiving a wireless signal;
wherein the wireless signal comprises an indicator indicative of the distance of the control device relative to the disinfection location;
wherein the controller is configured to determine a distance of the control device relative to the disinfection location based on said indicator.

10. The control device according to claim 9, wherein the indicator comprises at least one of: a signal strength, distance data, location information.

11. The control device according to claim 1, wherein the user interface is a touchscreen display.

12. The control device according to claim 1, wherein the operational parameter of the ultraviolet light-based disinfection device comprises one of: an on/off condition; a spectral distribution of light; a type of ultraviolet light; a light intensity; a duration of UV-light emittance; a beam width; a beam direction.

13. The control device according to claim 1, wherein the user interface element comprises a button, a rotary knob, or a slider.

14. A method of controlling a ultraviolet light-based disinfection device arranged at a disinfection location with a control device, wherein the control device is a portable device;
wherein the control device comprises an user interface comprising a user interface element, wherein the user interface element is configured to control an operational parameter of the ultraviolet light-based disinfection device by providing a degree of control in controlling said operational parameter;
wherein the method comprises:
(i) determining a spatial relation of the control device relative to the ultraviolet light-based disinfection device and/or the disinfection location, and to
(ii) changing the degree of control of the user interface element in controlling said operational parameter based on said spatial relation;
wherein the spatial relation is a distance of the control device relative to the disinfection location.

15. A system comprising an ultraviolet light-based disinfection device arranged at a disinfection location and a control device according to claim 1, wherein the control device is arranged to control the ultraviolet light-based disinfection device.
